(19) United States
(12) Patent Application Publication
(10) Pub. No.: US 2015/0232220 A1
(43) Pub. Date: Aug. 20, 2015

(54) Adult Incontinence Packages, Displays, and Advertisements

(71) Applicant: The Procter & Gamble Company, Cincinnati, OH (US)

(72) Inventors: Christopher Michael KEITH, Cincinnati, OH (US); Karen Rachel MARTIN, Cincinnati, OH (US)

(21) Appl. No.: 14/621,540

(22) Filed: Feb. 13, 2015

Related U.S. Application Data
(60) Provisional application No. 61/939,905, filed on Feb. 14, 2014.

(51) Int. Cl. B65B 61/26; A61F 13/551; B65B 5/06

(52) U.S. Cl. CPC B65B 61/26; B65B 5/06; A61F 13/551; A61F 13/55105

(57) ABSTRACT

A method of transforming an unprinted package to a printed package by printing graphics of at least two adults on a face of a package. A method of transforming an unprinted sheet to a printed sheet by printing graphics of at least two adults on the sheet. A package comprising indicia of two adults in close proximity and a plurality of absorbent articles. A shelf display comprising indicia of two adults in close proximity and a plurality of absorbent articles.

### CROSS REFERENCE TO RELATED APPLICATIONS

This application claims the benefit, under 35 USC 119(e), to U.S. Provisional Patent Application No. 61/939,905 filed on Feb. 14, 2014, which is herein incorporated by reference in its entirety.

### FIELD OF THE DISCLOSURE

The present disclosure generally relates to adult incontinence. And, more particularly, the present disclosure relates to packaging, shelf displays, and/or advertisements related to adult incontinence products.

### BACKGROUND OF THE DISCLOSURE

Taped diapers normally are packaged in open form, such that the waist opening and leg openings are not formed. Taped diapers normally have fastening components on opposing ears or side panels and require both hands of the caregiver to fasten the diaper around the child. Normally, taped diapers are applied to the child in the supine position.

It is critical to convey that a user of adult incontinence (AI) products can continue to connect with others in her life, even in this typically extremely isolating condition, due to the confidence she has from her AI product, such as her AI pant.

Research illustrates how powerful it can be when a person realizes that she's not alone in experiencing incontinence, and that there are others like her that have the condition. Once she finds someone like herself that suffers, she opens up, feels reassured, and feels empowered in her life rather than the typical feelings of isolation, despair, and struggle that she associates with AI. The use of illustrating two adults on a package or an advertisement symbolizes that she is not alone in her experience and that with certain AI products, she can continue to live a social life, remaining in close relationship with cherished friends and family. The same of course may be true with males.

Further, the AI product category is extremely difficult for adults to shop. Often consumers suffer from embarrassment and social isolation due to their condition, which makes their ability to shop the category extremely difficult. There are key functional needs that an AI product must address to enable consumers to be able to socially engage, but consumers are often unaware of these functional benefits even when offered as they do not spend adequate time studying the package. What is needed is an ability to communicate these functional benefits to the consumer so that she/he may be able to select the product that can meet those needs but not via words and icons that impact the discreteness with which they need to shop the category. This may be accomplished by the use of indicia on the package or a primary selling element of two women/men within close proximity to each other, without the need for packages or advertisements to describe multiple functional benefits of the product contained in the package, possibly including odor control, leakage prevention, product quietness, and product thinness. This utilizes our ability to quickly connect the crucial absorbent product features via the ultimate functional indicator possible for consumers—social acceptance. It may alternatively be desirable to use indicia of an adult male and female in close proximity to each other.

The product being advertised, however, needs to perform at a level consistent with the new promise of continuing with her social life in a normal way. If the product fails, the illustrations of two adults on the package is without use. This is a problem with many of the offerings today, they don't perform adequately to instill confidence. Most AI products on the market today don't even have an upstanding leg cuff like the ones commonly used for baby diapers.

It is an object of the present disclosure to teach how to transform and to provide a package and/or an advertisement to functionally communicate the high performance absorbent articles in the package.

### SUMMARY OF THE DISCLOSURE

In one embodiment, a method of transforming a package is disclosed, wherein the method may comprise the step of transforming an unprinted package to a printed package by printing graphics of at least two adults on a face of a package and also may comprise the step of transforming the printed package by filling the package with a plurality of adult incontinence absorbent articles relating to the printed graphics. The absorbent articles of the package may each comprise absorbent members, wherein at least one of the absorbent members has a body fluid absorption rate greater than 3 g/sec. Further, each of the absorbent articles may comprise leg cuffs. The at least two adults may be depicted as smiling or laughing.

In another embodiment, the present disclosure includes a method of transforming an advertisement, the method may comprise the step of transforming an unprinted advertisement to a printed advertisement by printing graphics of at least two adults on a front face of an advertisement. The method may also include relating the advertisement to a particular brand of adult incontinence pants and distributing the advertisement.

In another embodiment, the present disclosure includes a method of advertising adult incontinence products, the method may comprise the steps of showing two women together in a smiling or laughing poses, showing the two women below their waistlines, and associating the two women with a brand and sub-brand of product. The product may be an adult incontinence pant.

In another embodiment, the present disclosure includes a package that may comprise indicia of at least two adults on a face of the package. And, the package may also comprise a plurality of adult incontinence absorbent articles. The plurality of absorbent articles may each comprise absorbent members, wherein at least one of the absorbent members comprises less than 10% airfelt. Each of the absorbent articles may comprise standing leg cuffs. And, the at least two adults may be shown, at least in part, below their waistlines.

In yet another embodiment, the present disclosure includes a package that may comprise indicia of at least two adults on a face of the package and a plurality of adult incontinence absorbent articles. The plurality of absorbent articles may each comprise absorbent members, wherein at least one of the absorbent members comprises has an intake factor greater than 3 cc. The plurality of adult incontinence articles may be pre-closed. And, the package may not illustrate absorbent member or leg cuff features.

In yet another embodiment, the present disclosure includes a package that may comprise indicia of at least two adults on a face of the package and a plurality of adult incontinence absorbent articles. The at least two adults may be in close proximity and may be depicted as being in motion. The plurality of adult incontinence articles may be pre-closed. And, the package may not illustrate absorbent member or leg cuff features.

In another embodiment, the present disclosure includes a shelf display that may be incorporated into a larger shelf display or as a stand-alone display that may comprise indicia of at least two adults on a consumer-facing portion of the display and the display may comprise a plurality of adult incontinence absorbent articles in a plurality of packages. The at least two adults illustrated may be in close proximity and may be depicted as being in motion. The plurality of adult incontinence articles may be pre-closed. And, the packages may not illustrate absorbent member or leg cuff features.

### BRIEF DESCRIPTION OF THE DRAWINGS

FIGS. 1-4 illustrate advertisements comprising two adult women in close proximity.

FIG. 5 illustrates a feminine design.

FIGS. 6-12 illustrate shelf displays comprising a plurality of absorbent articles in a plurality of packages, wherein the shelf displays comprise images of two adult women.

FIGS. 13 and 14 illustrate packages of absorbent articles comprising two adult women on faces of the packages.

FIGS. 15 and 16 illustrate arrays of packages of absorbent articles, including packages of AI pants, pads, and liners.

FIG. 17 illustrates an absorbency scale from a portion of a package of absorbent articles.

### DETAILED DESCRIPTION OF THE DISCLOSURE

Non-limiting examples of disposable absorbent articles of the present disclosure include diapers, training pants, and adult incontinence products, and feminine hygiene products. Said disposable absorbent articles may be packaged, such that each package comprises a plurality of absorbent articles. The packages and/or advertisements may be printed or otherwise depict two adults.

### Absorbent Article

In one embodiment, an absorbent article may comprise a chassis comprising a topsheet, a backsheet, and an absorbent core disposed at least partially between the topsheet and the backsheet. The absorbent chassis may comprise a waistband, leg cuffs and or elastic strands. Flaps comprising fastening components may be attached to or integral with the chassis. Exemplary taped and pant-style diapers, as well as pads and liner-type articles are disclosed U.S. application Ser. No. 61/931229, filed Jan. 24, 2014.

A chassis of the absorbent article may comprise a topsheet. Suitable apertured films that may be used as or in combination with the topsheet are described in U.S. Pat. Nos. 3,929,135, 4,324,246, 4,342,314, 4,463,045, 5,006,394, 5,628,097, 5,916,661, 6,545,197, and 6,107,539.

Examples of suitable topsheet lotions include, but are not limited to, those described in U.S. Pat. Nos. 5,607,760; 5,609,587; 5,635,191; 5,643,588; and 5,968,025, and as described in U.S. Application Ser. No. 61/391,353.

The chassis of the absorbent article may comprise an absorbent core (often referred to as an absorbent assembly, structure, or member). In one embodiment, suitable absorbent cores may comprise cellulosic airfelt material. For instance, such absorbent cores may comprise less than about 40%, 30%, 20%, 10%, 5%, or even 1% of the cellulosic airfelt material as determined by weight. Additionally, such an absorbent core may be primarily comprised of an absorbent gelling material in amounts of at least about 60%, 70%, 80%, 85%, 90%, 95%, or even about 100% as determined by weight. Furthermore, a portion of the absorbent core may comprise a microfiber glue (if applicable). Such absorbent cores, microfiber glues, and absorbent gelling materials are described in U.S. Pat. Nos. 5,599,335; 5,562,646; 5,669,894; 6,790,798; and 7,521,587 and in U.S. Pat. Publ. No. 2004/0158212.

In one embodiment, the absorbent article of the present disclosure, and particularly, a portion where the absorbent member is disposed, may have a rate a body fluid absorption rate greater than 3 g/sec according to U.S. Pat. No. 6,649,810. According to U.S. Pat. No. 6,649,810, the expression “the portion (of the absorbent article) where the absorbent member is disposed” is intended to mean the portion occupied by the absorbent member when the absorbent article is flatly unfolded and seen in its plan view.

In one embodiment, the absorbent structure may have an intake factor greater than 3 cc, according to U.S. Pat. No. 7,073,373, wherein the intake factor is defined as the absorbent core permeability divided by the normalized retention capacity (which is defined by the Retention Capacity Test—also according to U.S. Pat. No. 7,073,373).

In one embodiment, the absorbent composite has a body fluid absorption greater than 75 g/100 cm², according to U.S. Pat. No. 6,649,810.

In one embodiment, a target location of the absorbent article may have a wicking value greater than 36%, according to U.S. Pat. No. 6,383,960.

In one embodiment, the absorbent article may have a bending stiffness between 0.05-1.0 gf, according to U.S. Pat. No. 5,810,796.

In one embodiment, the absorbent article may have a crotch fluid absorption rate greater than 3g/sec according to U.S. Pat. No. 6,649,810.

In one embodiment, a freeze-dried composite of the absorbent composite may have an intake rate of at least about 1.9 cubic centimeters (cc) of liquid/second at 80% composite saturation according to U.S. Pat. No. 6,689,934.

The chassis of the absorbent article may comprise leg cuffs. Suitable elasticized leg cuffs 147 may comprise those described in U.S. Pat. Nos. 3,860,003; 4,909,803; 4,695,278; 4,795,454; 4,704,115; and 4,909,803; and U.S. Pat. Publ. No. 2009/0312730.

The chassis of the absorbent article may comprise suitable elasticized waistbands may be constructed in a number of different configurations including those described in U.S. Pat. Nos. 4,515,595 and 5,151,092.

Flaps may be integral with or joined to the chassis. The flaps may be permanently or refastenably joined to each other or to another edge of the chassis to form waist and leg openings. The flaps may be engaged to form said waist and leg openings when the absorbent articles are in the package—such that the articles are pre-closed or pre-fastened in the package.

Flaps may comprise fastening components. Some exemplary fastening components are disclosed in U.S. Patent Nos. 3,848,594; 4,662,875; 4,846,815; 4,894,060; 4,946,527; 5,151,092; 5,221,274; and 6,432,098. The fastening system may also include primary and secondary fastening components, as disclosed in U.S. Pat. No. 4,699,622.

The absorbent article may comprise “wings” that are intended to wrap the edges of the wearer's undergarments in the crotch region and/or affix the article to the undergarment to avoid poor folding and premature detachment. Exemplary absorbent articles comprising wings are disclosed in U.S. Pat. No. 8,039,685.

Various suitable belt-like flap configurations can be found in U.S. Pub. No. 2013-0211363.

Absorbent articles of the present disclosure may comprise odor neutralizing material(s). Suitable odor neutralizing materials are disclosed in U.S. Pub. No. 2008-0249490 and European Patent No. 1886698.

The product features/components that enable a product to reliably allow its wearer to be in close proximity to others without fearing detection that the wearer is indeed wearing an adult incontinence product is the goal. For instance, the article should allow for the wearer to join in activities and to otherwise socialize without the article being noisy due to the crinkling of the backsheet, without being thick due to the core, without allowing the escape of odor, without allowing liquids to leak from it, etc.

### Package

The disposable absorbent articles may be packaged, such that each package comprises a plurality of disposable absorbent articles.

The package may comprise a film and/or paper such as cardboard.

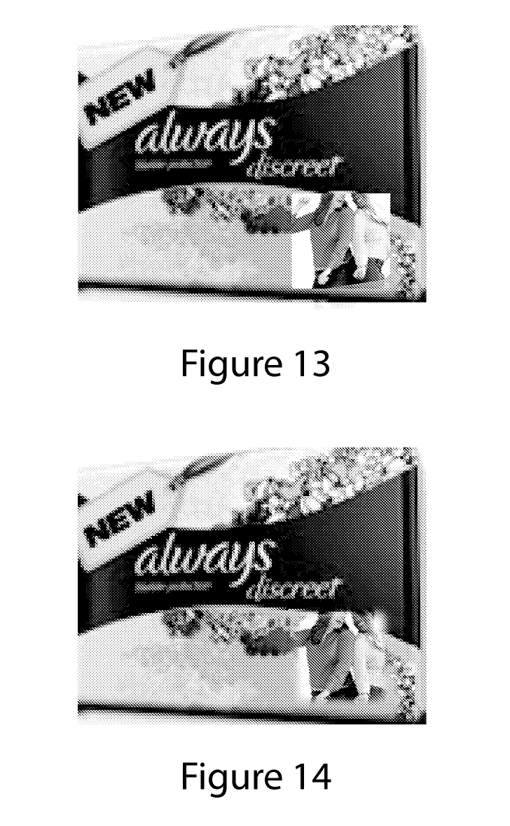

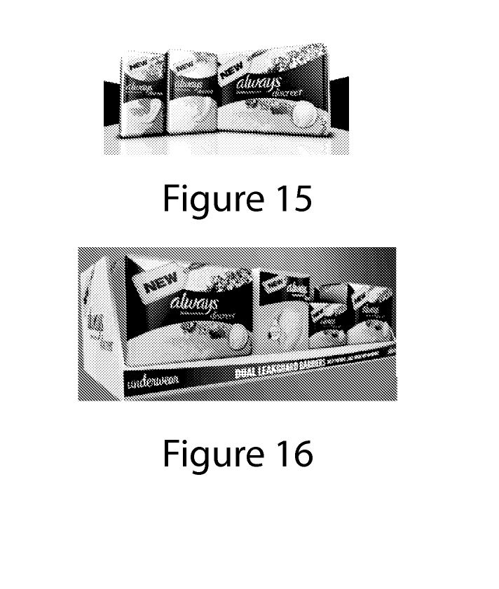

One or more faces of the package may comprise indicia of two or more adults for the purpose of communicating that it is okay to be social while being incontinent. For example, a package face, such as the buyer facing face as the package is on a shelf) may comprise two adult females in poses representative of a cheerful mood (see FIGS. 13 and 14). Two adults laughing together communicates that it is okay to enjoy the same activities as one did before they were incontinent because the absorbent articles inside the package can adequately protect the wearer from revealing her/his incontinence condition. In this way, there is a functional relationship between the indicia on the package and/or on advertisements (see below).

Further regarding the functional relationship, the confidence communicated to the wearer is related to the product features including one or a combination of leg cuffs and absorbent cores/articles having one or more of the following performance parameters, including the above-described intake factors, body fluid absorption rates, a body fluid absorptions, crotch fluid absorption rates, intake rates at 80%, and wicking values.

Further, the functional relationship can be drawn between confidence and discreteness of the absorbent article, including an article that is thinner because it utilizes an airfelt free core. Additionally, discreteness may be achieved by having the above-described bending stiffness.

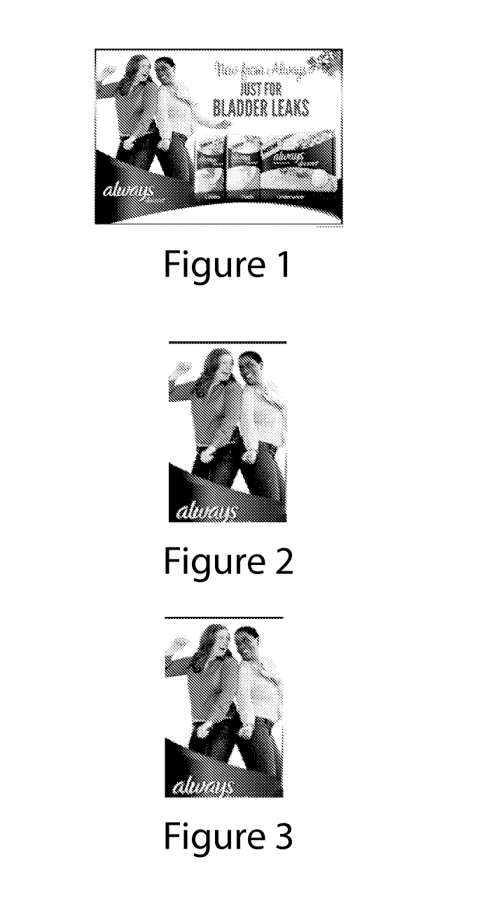

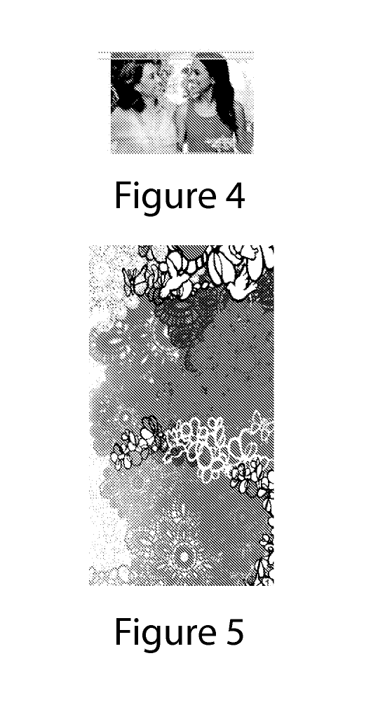

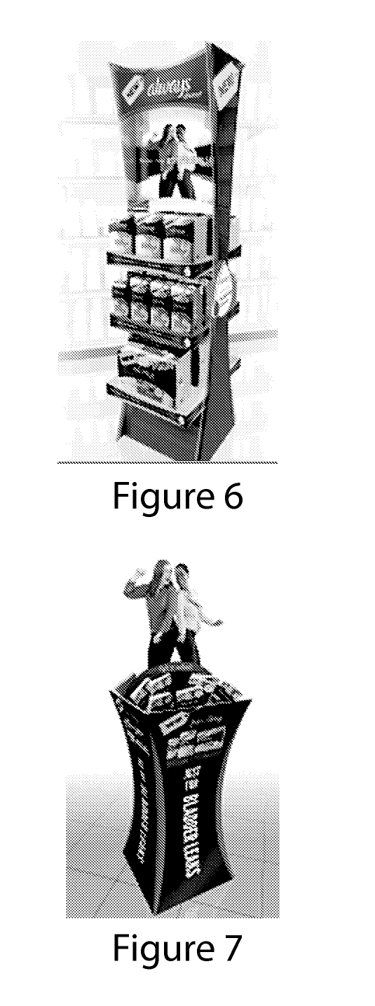

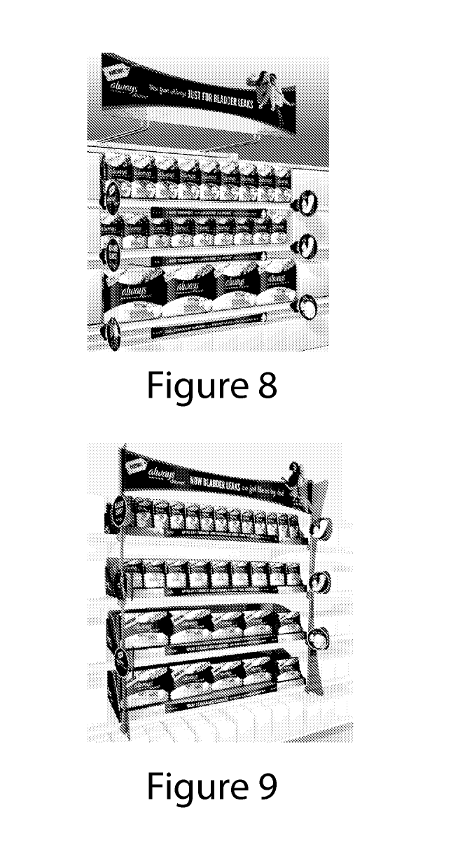

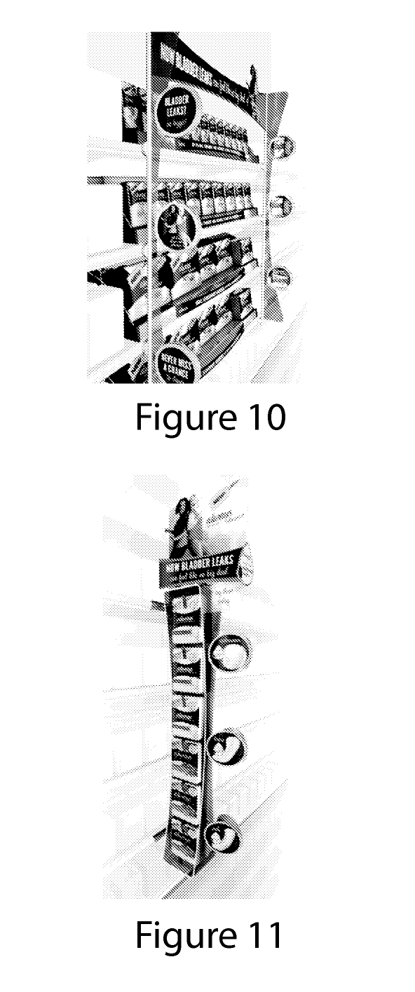

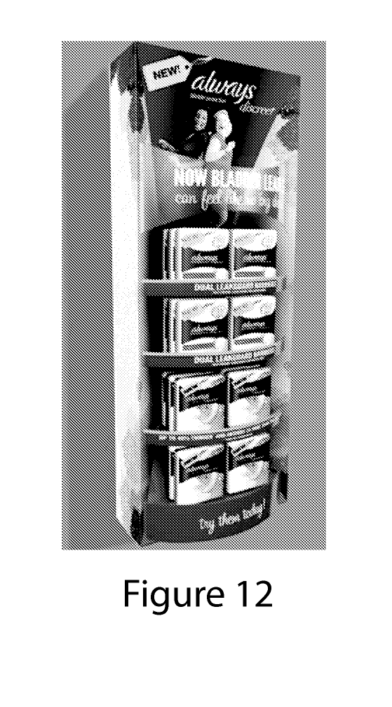

Confidence may be further communicated and the functional relationship between the capability of the articles to enable regular activities of the wearer may be achieved by graphically representing physical movements of the adults on the package, including dancing, picnicking, attending a play or a concert, bowling, jogging, lounging at a bar, hunting, or some other social event (see FIGS. 1-4 and 6-14). In this way, the jovial spirit graphically represented by two adults in such a setting or at such an activity communicates the functionality of the article within the package, without the need for additional descriptive claims (such as traditional leak prevention claims).

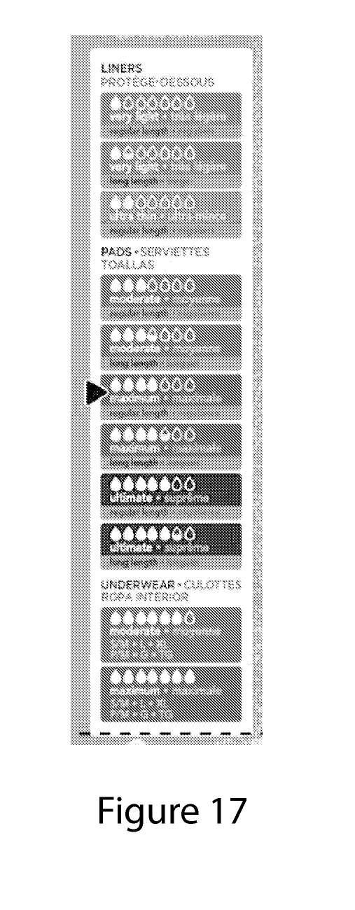

The package may further comprise a representation of absorbency and size. Regarding absorbency, the package may comprise more or less droplets versus higher or lower absorbency offerings (see FIG. 17). This representation may be placed on a face of the package opposite the imagery of the two adults.

In one embodiment, two women may be in back-to-back poses to show that a woman wearing adequately designed AI products can feel confident that her condition will stay private, even in close proximity to others. In such an embodiment, the two women may be in physical contact, such that their shoulders and/or buttocks areas may be touching.

In one embodiment, two women may be shown in poses depicting dynamic movement to demonstrate that she can move with ease with the properly designed absorbent article, confidently enjoying even this “torture test” situation.

In one embodiment, two women are shown below their waistlines to illustrates that the AI products can remain discreet underneath clothing. It may be implied that the women are wearing AI pants, for instance, by placing such imagery on the package of a brand and product known to be AI pants (e.g., Always Discreet or Depend Silhouette). Alternatively, such may be implied by imagery on an advertisement referring to the brand and product known to be AI pants.

It may be desirable to consistently use of two women throughout all elements of advertising (including TV, posters, flyers, packages, etc.) to achieve an iconic visual that is holistic and recognizable as wearers see a particular brand/sub-brand or a particular manufacturer.

### Advertisement

The same or similar indicia on the package above in the package section may be illustrated on advertisements, such as print ads, television commercials, mailings, flyers, posters, store shelving, newspapers, magazines, etc.

### Array

Packages of absorbent articles may be arranged in an array, such that a package as described above (where the package comprising a plurality of absorbent articles may comprise indicia of at least two adults) may be placed adjacent packages of the same brand and/or the same manufacturer that do not comprise such indicia. For instance, a package of adult incontinence pants comprising indicia of two adults on the package may be placed in an array with packages of pads and packages of liners that do not comprise such “two adult” imagery.

However, other embodiments of the present disclosure may comprise packages of adult incontinence articles, pads, and liners, each in packages comprising graphics of two adults. As above, the adults may be laughing or smiling, and may be engaged in an activity. In this embodiment, the two adults on the package of AI pants may be different than the two adults on each of the pads and liners packages.

In another embodiment, different offerings (e.g., absorbencies, sizes, etc.) of the AI pants may comprise different packages comprising different sets of at least two adults. For instance, a smaller size of AI pants may exhibit thinner adults on the packages and may be situated in proximity to a package of larger sized AI pants that illustrates heavier adults (relative to the thinner adults referenced).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of transforming a package, the method comprising the steps of:
transforming an unprinted package to a printed package by printing graphics of at least two adults on a face of a package;
further transforming the printed package by filling the package with a plurality of adult incontinence absorbent articles relating to the printed graphics;
wherein the absorbent articles of the package each comprise absorbent members, and wherein at least one of the absorbent members has a body fluid absorption rate greater than 3 g/sec;
wherein each of the absorbent articles comprise leg cuffs; and
wherein the at least two adults are depicted as smiling or laughing.

2. The method of claim 1, wherein the package comprises graphics illustrating a likeness of the cuffs.

3. The method of claim 1, wherein the package comprises graphics illustrating a likeness of the absorbent member.

4. The method of claim 1, wherein the absorbent articles are in closed form.

5. The method of any of claim 1, wherein the at least two adults are two women.

6. The method of any of claim 1, wherein the at least two adults are two men.

7. The method of any of claim 1, wherein the at least two adults are one man and one woman.

8. The method of any of claim 1, wherein the at least two adults are depicted as smiling.

9. The method of any of claim 1, wherein the at least two adults are depicted as laughing.

10. The method of claim 1, wherein the at least two adults are engaging in an activity selected from the group consisting of running, walking, jogging, hiking, camping, biking, bowling, fishing, drinking, talking, laughing, joking, storytelling, eating, golfing, playing soccer, and combinations thereof.

11. The method of claim 1, wherein the package further comprises graphics representing the absorbent capacity of the plurality of absorbent articles within the package.

12. The method of claim 1, wherein the graphics representing the absorbent capacity of the plurality of absorbent articles within the package comprise droplets.

13. The method of claim 1, wherein more droplets equates to a greater absorbent capacity of the absorbent articles within the package versus other packages comprising other absorbent capacities from the same brand and sub-brand.

14. The method of claim 1, wherein the leg cuffs are elevated in a z direction when the article is unfolded and laid flat.

15. The method of claim 1, wherein at least one of the absorbent articles of the package has a bending stiffness between 0.05-1.0 gf.

16. The method of claim 1, wherein at least one absorbent composite of at least one of the absorbent articles of the package has a body fluid absorption greater than 75 g/100 cm².

17. The method of claim 1, wherein at least one absorbent article of the package has a crotch fluid absorption rate greater than 3 g/sec.

18. The method of claim 1, wherein at least one absorbent member of at least one of the absorbent article of the package has an intake factor greater than 3 cc.

19. The method of claim 1, wherein at least one freeze-dried composite of at least one absorbent composite of at least one absorbent article of the package has an intake rate at 80% saturation of greater than 1.9 cc.

20. The method of claim 1, wherein at least one a target location of at least one of the absorbent articles of the package has a wicking value greater than 36%.
